(19) United States
(12) Patent Application Publication
(10) Pub. No.: US 2014/0018681 A1
(43) Pub. Date: Jan. 16, 2014

(54) ULTRASOUND IMAGING BREAST TUMOR DETECTION AND DIAGNOSTIC SYSTEM AND METHOD

(71) Applicant: NATIONAL TAIWAN UNIVERSITY, Taipei (TW)

(72) Inventors: RUEY-FENG CHANG, Taipei (TW); CHIUN-SHENG HUANG, Taipei (TW); YI-HONG CHOU, Taipei (TW); YEUN-CHUNG CHANG, Taipei (TW); WEI-WEN HSU, Taipei (TW); YI-WEI SHEN, Taipei (TW); YAN-HAO HUANG, Taipei (TW)

(73) Assignee: NATIONAL TAIWAN UNIVERSITY, Taipei (TW)

(21) Appl. No.: 13/729,444

(22) Filed: Dec. 28, 2012

(30) Foreign Application Priority Data
Jul. 10, 2012 (TW) 101124714

(51) Int. Cl. A61B 8/08

(52) U.S. Cl. CPC A61B 8/0825
USPC 600/443

(57) ABSTRACT

An ultrasound imaging breast tumor detection and diagnostic system and method is disclosed. The method uses the system to acquire a plurality of 3D breast ultrasound images, and then to cut out multiple regions from the 3D breast ultrasound images using a 3D means shift algorithm, and then to acquire the mean grayscale value (MGV) of each region, and then to classify the regions to groups subject to the mean grayscale value (MGV), and to merge each of the regions of the darkest group with adjacent regions of the similar grayscale into a respective suspicious tumor tissue full region, and then to recognize each suspicious tumor tissue full region to be a tumor tissue region or non-tumor tissue region. Thus, using region as the basic computing unit, tumor tissues are quickly recognized from the 3D breast ultrasound images.

### 1. FIELD OF THE INVENTION

The present invention relative to breast tumor detection and diagnostic technology and more particularly, to an ultrasonic imaging breast tumor detection and diagnostic system for rapidly detecting breast tumor tissues from ultrasound images. The invention relates also to detection and diagnosis of breast tumor using the ultrasonic imaging breast tumor detection and diagnostic system.

### 2. BACKGROUND OF THE INVENTION

Breast cancer has become one of the commonest cancers for women. According to clinical experiences, the cure rate would be rather high if a patient is medically treated right after being diagnosed with breast cancer at an early stage.

Currently, the ultrasound breast examination is one of major techniques for breast lesion checkup because of its advantages such as non-radioactivity, non-invasiveness, and harmlessness to breast tissues, accurate positioning, convenience and ease of use. Most of all, it is less costly compared to CT and MRT. Moreover, ultrasound breast examination is suitable for Asian women because their breast tissues are denser.

Further, during a breast ultrasound scanning operation, a large amount of breast ultrasound images can be synchronously recorded by a breast ultrasound imaging system. Further, a breast ultrasound imaging system can detect the presence of tumor tissues by means of analysis of breast ultrasound imaging contents, thereby reminding the doctor of the situations.

The breast ultrasound image analysis programs of conventional breast ultrasound imaging systems are mostly pixel-based, i.e., using pixel as the basic computing unit. However, a high-quality breast ultrasound image is often more than a million pixels, and its operation is very impressive, so that the breast ultrasound image analysis program must spend a lot of time.

In view of this, the present invention provides an ultrasonic imaging breast tumor detection and diagnostic system and method using region as the basic computing unit. This region-based computing method not only can greatly reduce the computational complexity and quickly detect tumor tissues, but also can effectively eliminate speckle noise from the images.

### SUMMARY OF THE PRESENT INVENTION

It is one object of the present invention to provide an ultrasonic imaging breast tumor detection and diagnostic system and method, which is configured to cut out a plurality of regions from the scanned 3D breast ultrasound images and to analyze 3D breast ultrasound images using every region as a basic computing unit, thereby greatly reducing the amount of computation and rapidly recognizing tumor tissues from the 3D breast ultrasound images.

It is another object of the present invention to provide an ultrasonic imaging breast tumor detection and diagnostic system and method, which achieves image smoothing effects by effectively removing speckle noise from the scanned 3D breast ultrasound images using a 3D means shift algorithm.

It is still another object of the present invention to provide an ultrasonic imaging breast tumor detection and diagnostic system and method, which is used for classifying each tumor tissue region recognized from the 3D breast ultrasound images by using the BI-RADS (breast imaging reporting and data system) assessment category scale so as to the doctor to be clear of the benign and malignant status of each tumor tissue region.

It is still another object of the present invention to provide an ultrasonic imaging breast tumor detection and diagnostic system and method, which provides a tumor map and marks all suspicious tumor tissue full regions on the tumor map in different colors subject to the BI-RADS assessment categories of the tumor tissue regions so as to facilitate doctor observing the tumor tissue distribution and determining the severity of the tumor tissue.

It is still another object of the present invention to provide an ultrasonic imaging breast tumor detection and diagnostic system and method, which is used for classifying each tumor tissue region recognized from the 3D breast ultrasound images by using the BI-RADS assessment-category scale and calculating the position and size of each tumor tissue region so that the doctor easily achieve the breast diagnostic report recording work.

To achieve above objects, the present invention provides an ultrasonic imaging breast tumor detection and diagnostic system, comprising: an image acquisition module adapted to acquire a plurality of 3D breast ultrasound images; an image segmentation module connected to the image acquisition module and used to receive the 3D breast ultrasound images so as to cut out a plurality of regions from the 3D breast ultrasound images using a 3D means shift algorithm; a mean grayscale value acquisition module connected to the image segmentation module and used to acquire the mean grayscale value (MGV) of each region; a region classification module connected to the mean grayscale value acquisition module, the region classification module having set therein a plurality of groups for classifying the mean grayscale value (MGV) of each region to the corresponding group, so that the regions classified to the darkest group are considered as suspicious tumor regions; and a region merging module connected to the region classification module and used to respectively merge each of the regions of the darkest group with at least one adjacent region of the similar grayscale into a respective suspicious tumor tissue full region.

In one embodiment of the present invention, wherein the 3D means shift algorithm is used for clustering each of adjacent pixels with a respective similar grayscale value in the 3D breast ultrasound images into the same region.

In one embodiment of the present invention, wherein the region classification module is configured to employ a fuzzy c-means algorithm for classifying the mean grayscale value (MGV) of each region to groups.

In one embodiment of the present invention, further comprising a characteristic acquisition and analysis module connected to the region merging module, and used to acquire and analyze at least one tumor characteristic from each suspicious tumor tissue full region so as to recognize each suspicious tumor tissue full region to be a tumor tissue region or non-tumor tissue region.

In one embodiment of the present invention, wherein the characteristic acquisition and analysis module is used for classifying each recognized tumor tissue region by using the BI-RADS (breast imaging reporting and data system) assessment category scale.

In one embodiment of the present invention, further comprising a tumor marker module connected to the characteristic acquisition and analysis module, the tumor marker module providing a tumor map and adapted to mark all recognized tumor tissue regions on the tumor map according to their distribution in the position of breast.

In one embodiment of the present invention, wherein the tumor marker module is used for marking all recognized tumor tissue regions on the tumor map in different colors subject to the respective classified BI-RADS assessment categories.

In one embodiment of the present invention, wherein the tumor marker module is used for calculating the position and size of each recognized tumor tissue region based on the nipple position as a center.

In one embodiment of the present invention, further comprising a user interface connected to the tumor marker module, the user interface comprising a tumor map display zone and being adapted to display the tumor map on the tumor map display zone.

In one embodiment of the present invention, further comprising a user interface connected to the tumor marker module, the user interface comprising a tumor diagnosis zone adapted to display the diagnostic result of each recognized tumor tissue region in a list, the diagnostic result comprising the BI-RADS assessment category of each recognized tumor tissue region and the position and/or size of each recognized tumor tissue region.

In one embodiment of the present invention, wherein the diagnostic results of the recognized tumor tissue regions are listed in a predetermined order subject to the respective BI-RADS assessment categories and positions and/or sizes.

In one embodiment of the present invention, further comprising a user interface connected to the image acquisition module, the user interface comprising a breast scanning site selection zone and an ultrasound imaging display zone, the breast scanning site selection zone comprising a plurality of scanning site selection components, the 3D breast ultrasound image of the corresponding breast site be displayed on the ultrasound imaging display zone via the click of the specific scanning site selection component.

The present invention further provides an ultrasonic imaging breast tumor detection and diagnostic method, comprising the steps of: acquiring a plurality of 3D breast ultrasound images; cutting out a plurality of regions from the 3D breast ultrasound images using a 3D means shift algorithm; acquiring the mean grayscale value (MGV) of each region; setting a plurality of groups for classifying the mean grayscale value (MGV) of each region; classifying each region to the corresponding group subject to the mean grayscale value (MGV) of each region; and merging each of the regions of the darkest group with at least one adjacent region of the similar grayscale into a respective suspicious tumor tissue full region.

In one embodiment of the present invention, further comprising the steps of: acquiring at least one tumor characteristic from each suspicious tumor tissue full region; and analyzing the tumor characteristic of each suspicious tumor tissue full region to recognize each suspicious tumor tissue full region to be a tumor tissue region or non-tumor tissue region.

In one embodiment of the present invention, further comprising the step of performing a benign and malignant classification on each suspicious tumor tissue full region been recognized as a tumor tissue region by using the Bl-RADS assessment-category scale.

In one embodiment of the present invention, further comprising the step of marking all recognized tumor tissue regions on a tumor map.

In one embodiment of the present invention, wherein all recognized tumor tissue regions are marked on a tumor map in different colors subject to the respectively classified BI-RADS assessment categories.

In one embodiment of the present invention, further comprising the step of calculating the position and size of each recognized tumor tissue region.

### BRIEF DESCRIPTION OF THE DRAWINGS

FIG. 1 is a system block diagram of an ultrasound imaging breast tumor detection and diagnostic system in accordance with one preferred embodiment of the present invention.

FIG. 2 is a schematic drawing of a 3D breast ultrasound image in accordance with the present invention.

FIG. 3 is a schematic drawing illustrating a segmented region of a 3D breast ultrasound image in accordance with the present invention.

FIG. 4 is a schematic drawing illustrating a segmented region of a 3D breast ultrasound image after a classification procedure in accordance with the present invention.

FIG. 5 is a schematic drawing illustrating a classified region of a 3D breast ultrasound image after a merge procedure in accordance with the present invention.

FIG. 6 is a schematic drawing illustrating a classified region of a 3D breast ultrasound image after a tumor analysis procedure in accordance with the present invention.

FIG. 7 is a schematic drawing of the user interface of the ultrasound imaging breast tumor detection and diagnostic system in accordance with the present invention.

FIG. 8 is a flow chart of an ultrasound imaging breast tumor detection and diagnostic method in accordance with one preferred embodiment of the present invention.

### DETAILED DESCRIPTION OF THE PREFERRED EMBODIMENTS

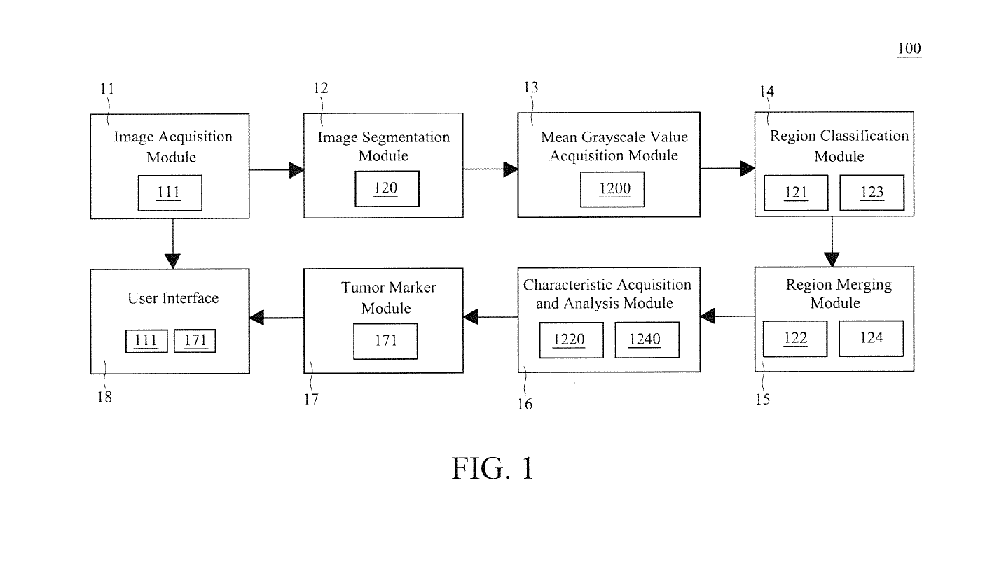

Please refer to FIG. 1, a system block diagram of an ultrasound imaging breast tumor detection and diagnostic system in accordance with one preferred embodiment of the present invention is shown. As illustrated, the ultrasound imaging breast tumor detection and diagnostic system 100 comprises an image acquisition module 11, an image segmentation module 12, a mean grayscale value acquisition module 13, a region classification module 14, and a region merging module 15.

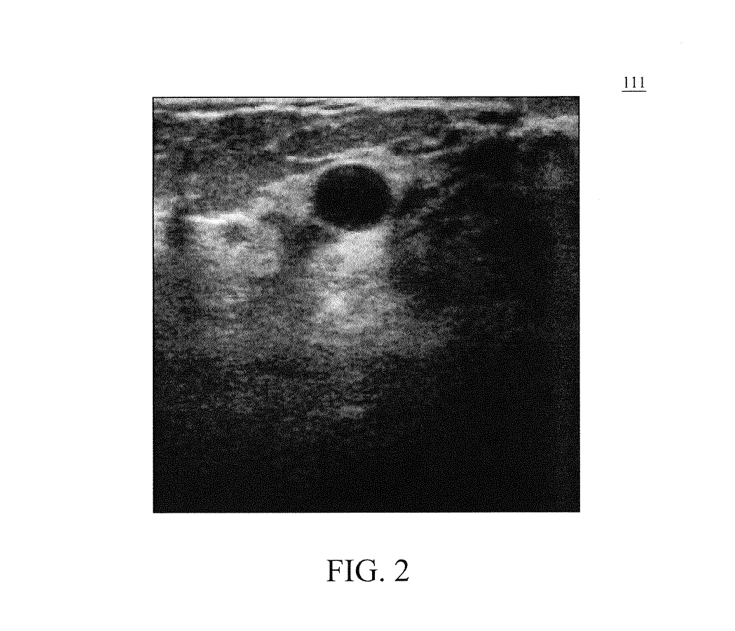

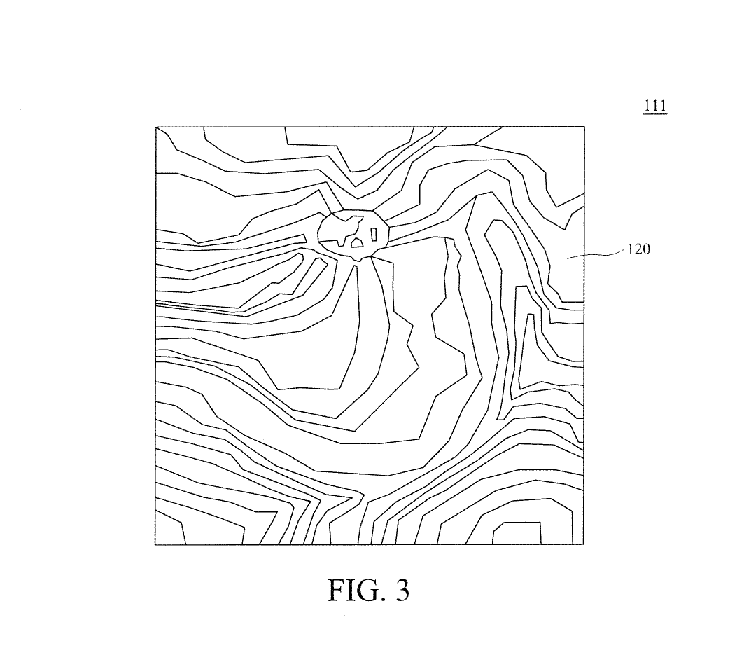

Firstly, an ultrasound probe performs the breast ultrasound scanning process on the breasts so as to acquire a continuous of 3D breast ultrasound images 111 by the image acquisition module 11, as shown in FIG. 2. The image segmentation module 12 is connected to the image acquisition module 11 to receive the 3D breast ultrasound images 111 and to cluster each of adjacent pixels with a respective similar grayscale value in the 3D breast ultrasound images into the same region by using a 3D means shift algorithm so that the 3D breast ultrasound images 111 can be segmented into multiple regions 120, as shown in FIG. 3.

The mean grayscale value acquisition module 13 is connected to the image segmentation module 12, and used for calculating the grayscale pixel mean value of every region 120 so as to acquire the mean grayscale value (MGV) 1200 of each region 120.

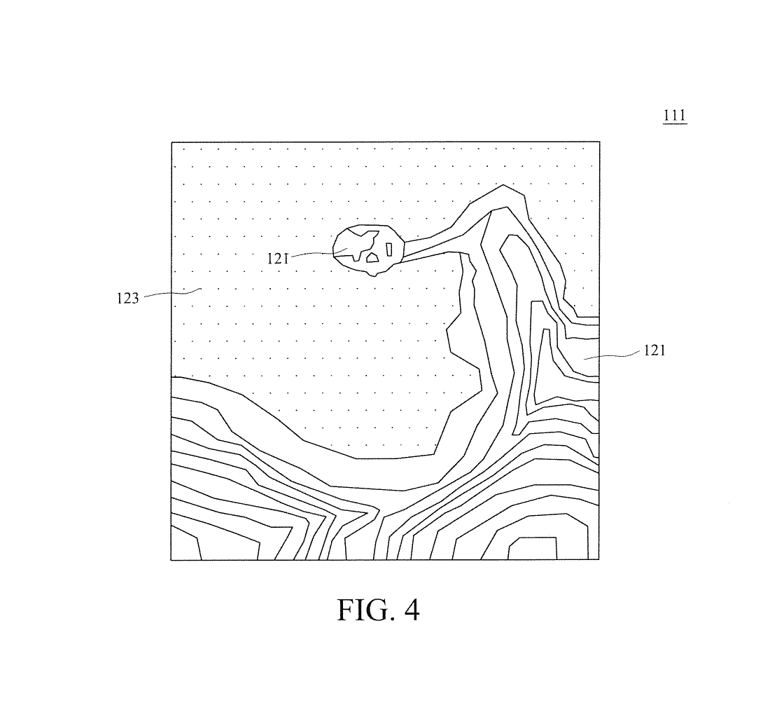

The region classification module 14 is connected to the mean grayscale value acquisition module 13, having set therein multiple, for example, 4 groups for classifying the mean grayscale value (MGV) 1200 of each region 120. Further, the region classification module 14 can be configured to employ a fuzzy c-means algorithm for classifying the mean grayscale value (MGV) 1200 of each region 120. As shown in FIG. 4, after region classification, 3D breast ultrasound images 111 can be classified into regions 121 of a first type and region 123 of a second type, wherein the regions 121 of the first type are classified to the darkest group: the region 123 of the second type is classified to the other group with brighter grayscale value. Further, in one embodiment of the present invention, the region 123 of the second type can be further processed through an image filtering process to remove the image contents. For general ultrasound scan, the color of a tumor tissue is more dark and deep than the color of a normal tissue. Thus, the regions 121 of the first type classified to the darkest group may be considered as a suspicious tumor region.

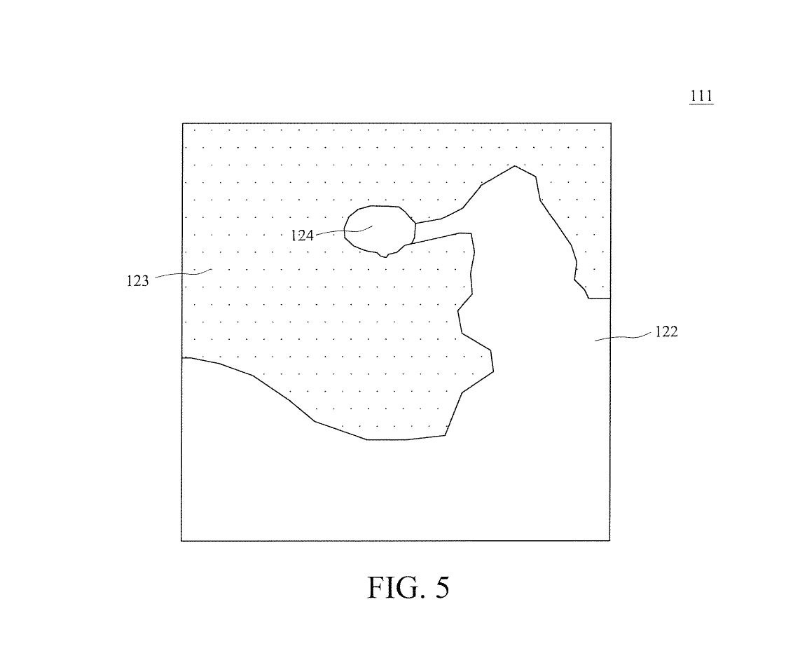

The region merging module 15 is connected to the region classification module 14, and used to respectively merge each of the regions 121 of the first type of the darkest group with at least one adjacent similar region 121 of the first type (for example: the difference between the mean grayscale values (MGV) 1200 of two regions 120 is within a predetermined threshold range) into a respective suspicious tumor tissue full region 122 [symbol] 124, so as to really cut out a suspicious tumor boundary, as shown in FIG. 5.

The ultrasound imaging breast tumor detection and diagnostic system 100 further comprises a characteristic acquisition and analysis module 16 connected to the region merging module 15, and used to acquire at least one tumor characteristic 1220 [symbol] 1240 from every suspicious tumor tissue full region 122 [symbol] 124, such as the region volume, the mean grayscale value, the standard deviation of grayscale value, and/or the grayscale difference between neighboring tissues, etc. Afterward, the characteristic acquisition and analysis module 16 analyzes the tumor characteristic 1220 [symbol] 1240 so as to recognize each suspicious tumor tissue full region 122 [symbol] 124 to be a tumor tissue region or non-tumor tissue region. In this example, the suspicious tumor tissue full region 124 is recognized as a tumor tissue region, and the suspicious tumor tissue full region 122 is recognized as a non-tumor tissue region.

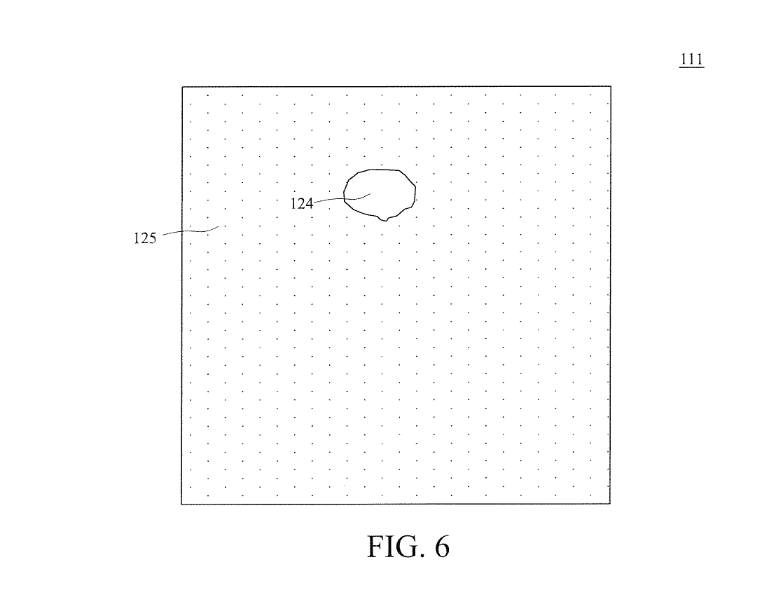

Further, as shown in FIG. 5 and FIG. 6, in one application example of the present invention, after the suspicious tumor tissue full region 122 was recognized as a non-tumor tissue region, an image filtering process is employed to remove image contents from the suspicious tumor tissue full region 122 and enabling the suspicious tumor tissue full region 122 to be combined with the other region 123 to form a non-tumor tissue region 125. Thus, analysis by the characteristic pickup and analysis module 16 to assist doctors to detect and diagnose the authenticity of the suspicious tumor tissue full regions 122 [symbol] 124 can effectively reduce the occurrence of too many non-tumor tissues to be erroneously diagnosed as a tumor.

Further, in one embodiment of the present invention, the characteristic acquisition and analysis module 16 further classifies every recognized tumor tissue region 124 by using the BI-RADS (breast imaging reporting and data system) assessment category scale, for example, BI-RADS 0-6. Subject to the BI-RADS classified assessment category, the doctor is clear of the benign and malignant status of every recognized tumor tissue region 124. However, it is to be understood that, except the method of using the breast imaging reporting and data system (BI-RADS) assessment-category scale to classify every recognized tumor tissue region 124, the doctor can judge the benign and malignant status of every recognized tumor tissue region 124 or correct the classification result of the characteristic acquisition and analysis module 16 subject to his (her) medical experience so as to enhance diagnosis accuracy. As stated above, the ultrasound imaging breast tumor detection and diagnostic system 100 of the invention uses every region 120 as a basic computing unit for analysis of 3D breast ultrasound images 111, it not only can greatly reduce the computation to detect and cut out tumor tissues from the 3D breast ultrasound images 111 but also achieve image smoothing effects by effectively removing speckle noise from the 3D breast ultrasound images 111 by using the 3D means shift algorithm.

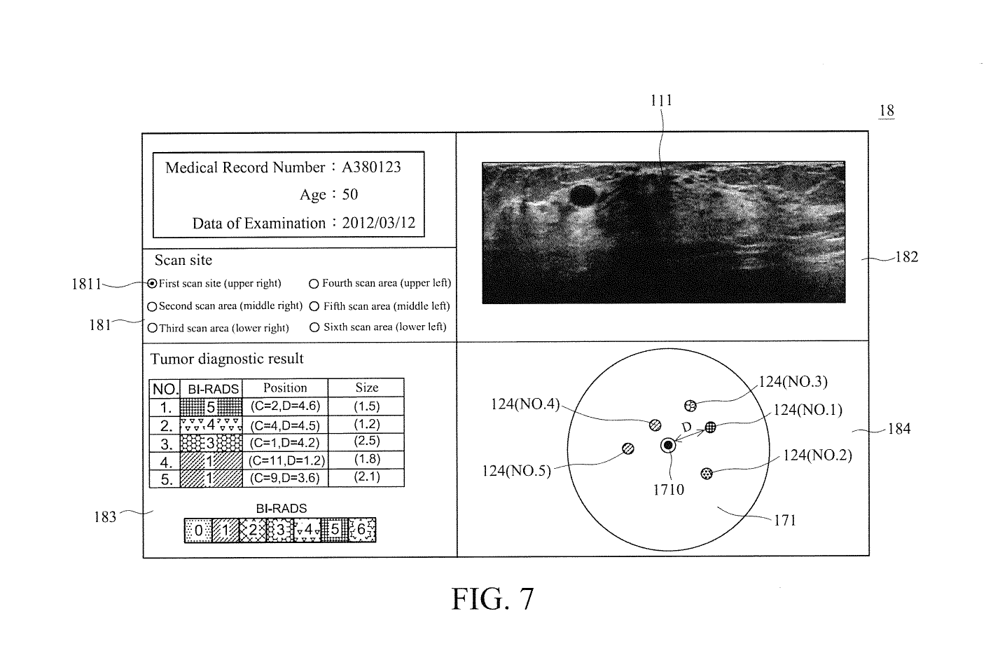

Referring to FIG. 7 and FIG. 1 again, a schematic diagram illustrating a user interface used for displaying the 3D breast ultrasound imaging and tumor diagnostic information. The ultrasound imaging breast tumor detection and diagnostic system 100 comprises a tumor marker module 17 and a user interface 18. The tumor marker module 17 is connected to the characteristic acquisition and analysis module 16. The user interface 18 is connected to the image acquisition module 11 and/or the tumor marker module 17.

The user interface 18 comprises a breast scanning site selection zone 181, an ultrasound imaging display zone 182, a tumor diagnosis zone 183, and a tumor map display zone 184.

Wherein, the breast scanning site selection zone 181 comprises a plurality of scanning site selection components 1811. During a breast ultrasound scanning operation, the doctor moves the ultrasound probe over selected breast site to start scanning breast ultrasound imaging, so that the image acquisition module 11 acquires a continuous of 3D breast ultrasound images 111 from every selected breast site. At this time, the continuous of 3D breast ultrasound images 111 acquired from every selected breast site is linked to a respective scanning site selection component 1811. Thus, the doctor can click one specific scanning site selection component 1811 and then view the continuous of 3D breast ultrasound images 111 of the corresponding breast site. For example, when the doctor clicks the first scanning site selection component 1811, the ultrasound imaging display zone 182 will display the 3D breast ultrasound images 111 of the upper right part of the breast.

The tumor marker module 17 provides a tumor map 171 that will be displayed on the tumor map display zone 184 of the user interface 18. The tumor marker module 17 marks all recognized tumor tissue regions 124 on the tumor map 171 in different colors subject to their original distribution location in the breast and their BI-RADS assessment categories. For example, every recognized tumor tissue region 124 classified as BI-RADS 0 is marked in brown, or purple for BI-RADS 1, blue for BI-RADS 2, green for BI-RADS 3, yellow for BI-RADS 4, orange for BI-RADS 5, and red for BI-RADS 6.

Further, after the tumor marker module 17 marks all recognized tumor tissue regions 124 on the tumor map 171, the position of every recognized tumor tissue region 124 is calculated and indicated by clockwise (clock; C) and distance (distance; D) based on the nipple 1719 position as a center. Further, the tumor marker module 17 will simultaneously calculate the size of every recognized tumor tissue region 124, for example, the maximum diameter of tumor.

Thereafter, the tumor diagnosis zone 183 of the user interface 18 displays the diagnostic result of every recognized tumor tissue region 124 in a list. The diagnostic result includes the BI-RADS assessment category of each recognized tumor tissue region 124 and the position and/or size of each recognized tumor tissue region 124. Further, the diagnostic results of the recognized tumor tissue regions 124 can be listed in a predetermined order subject to their BI-RADS assessment categories, their positions and/or sizes.

In the example shown in FIG. 7, the diagnostic results of the recognized tumor tissue regions 124 are sequentially arranged in accordance with their BI-RADS assessment categories. For example, the recognized tumor tissue regions 124 arranged in the first priority (No. 1) are classified to the BI-RADS 5; the recognized tumor tissue regions 124 arranged in the fifth priority (No. 5) are classified to the BI-RADS 1.

In accordance to the above-stated statement, the ultrasound imaging breast tumor detection and diagnostic system 100 of the present invention uses the tumor map 171 to show the location of every recognized tumor tissue region 124 in the breast and to mark the location with a corresponding color subject to its BI-RADS assessment category so as to facilitate the doctor observing the tumor tissue distribution and determining the severity of the tumor tissue. Further, with the diagnostic results indicated by the tumor diagnosis zone 183, the doctor can aware of the condition of the tumor tissue to be benign or malignant and the information of the position and size of the tumor tissue, easily achieving the breast diagnostic report recording work.

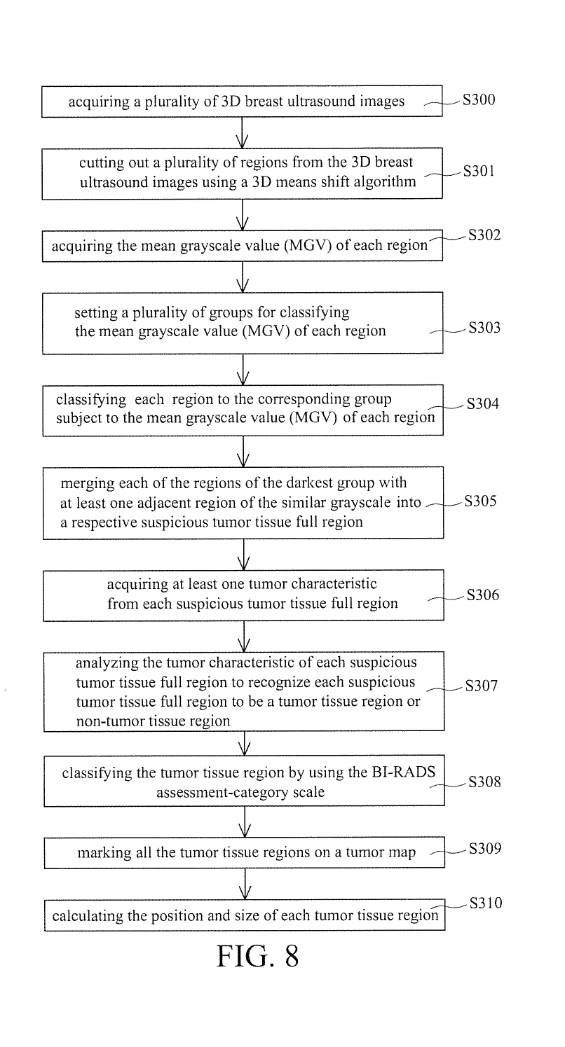

Referring to FIG. 8, there is shown a flow chart of an ultrasound imaging breast tumor detection and diagnostic method in accordance with one preferred embodiment of the present invention. Firstly, in step S300, the ultrasound probe performs the breast ultrasound scanning process on the breasts so as to acquire a continuous of 3D breast ultrasound images 111 by the image acquisition module 11, as shown in FIG. 2.

In Step 301, the image segmentation module 12 can cluster each of adjacent pixels with a respective similar grayscale value in the 3D breast ultrasound images into the same region by using a 3D means shift algorithm so that the 3D breast ultrasound images 111 can be segmented into multiple regions 120, as shown in FIG. 3.

In Step 302, the mean grayscale value acquisition module 13 can calculate the grayscale pixel mean value of every region 120 so as to acquire the mean grayscale value (MGV) 1200 of each region 120.

In Step 303, the region classification module 14 has set therein a plurality of groups for classifying the mean grayscale value (MGV) 1200 of each region 120.

In Step 304, the region classification module 14 can be used to classify the mean grayscale value (MGV) 1200 of each region 120 to the corresponding group so that the regions 120 of the 3D breast ultrasound images 111 having the darkest region color are classified to regions 121 of the darkest group and considered as suspicious tumor regions, as shown in FIG. 4.

In Step 305, the region merging module 15 is used to respectively merge each of the regions 121 of the first type of the darkest group with at least one adjacent similar region 121 of the first type into a respective suspicious tumor tissue full region 122 [symbol] 124, as shown in FIG. 5. In Step 306, the characteristic acquisition and analysis module 16 is used to acquire at least one tumor characteristic 1220 [symbol] 1240 from every suspicious tumor tissue full region 122 [symbol] 124.

In Step 307, the characteristic acquisition and analysis module 16 is used to judge each suspicious tumor tissue full region 122 [symbol] 124 to be a tumor tissue region or non-tumor tissue region by analyzing the respective tumor characteristics 1220 [symbol] 1240. Take FIG. 5 and FIG. 6 as an example, the suspicious tumor tissue full region 124 is recognized as a tumor tissue region and the suspicious tumor tissue full region 122 is recognized as a non-tumor tissue region. In Step 308, after the tumor tissue region 124 is recognized from the 3D breast ultrasound images 111, the characteristic acquisition and analysis module 16 further starts a benign and malignant classification of the recognized tumor tissue region 124 by using the BI-RADS assessment category scale.

In Step 309, the tumor marker module 17 marks all recognized tumor tissue regions 124 on the tumor map 171 in different colors subject to their original distribution location in the breast and their BI-RADS assessment categories so as to facilitate the doctor observing the tumor tissue distribution and determining the severity of the tumor tissue.

Finally, in Step 310, the tumor marker module 17 simultaneously calculates the size of each recognized tumor tissue region 124 when marking each recognized tumor tissue region 124 on the tumor map 171, and then to combine the BI-RADS assessment category, position and size of each recognized tumor tissue region 124 into a diagnostic result for reference by the doctor so that the doctor can easily achieve the breast diagnostic report recording work.

The present invention is not limited to the above-described embodiments. Various alternatives, modifications and equivalents may be used. Therefore, the above embodiments should not be taken as limiting the scope of the invention, which is defined by the appending claims.

What is claimed is:

1. An ultrasonic imaging breast tumor detection and diagnostic system, comprising:
an image acquisition module used to acquire a plurality of 3D breast ultrasound images;
an image segmentation module connected to said image acquisition module and used to receive said 3D breast ultrasound images so as to cut out a plurality of regions from said 3D breast ultrasound images using a 3D means shift algorithm;
a mean grayscale value acquisition module connected to said image segmentation module and used to acquire the mean grayscale value (MGV) of each said region;
a region classification module connected to said mean grayscale value acquisition module, said region classification module having set therein a plurality of groups for classifying the mean grayscale value (MGV) of each said region to the corresponding group, so that the regions classified to the darkest group are considered as suspicious tumor regions; and
a region merging module connected to said region classification module and used to respectively merge each of the regions of the darkest group with at least one adjacent region of the similar grayscale into a respective suspicious tumor tissue full region.

2. The ultrasonic imaging breast tumor detection and diagnostic system according to claim 1, wherein said 3D means shift algorithm is used for clustering each of adjacent pixels with a respective similar grayscale value in said 3D breast ultrasound images into the same region.

3. The ultrasonic imaging breast tumor detection and diagnostic system according to claim 1, wherein said region classification module is configured to employ a fuzzy c-means algorithm for classifying the mean grayscale value (MGV) of each said region to the corresponding group.

4. The ultrasonic imaging breast tumor detection and diagnostic system according to claim 1, further comprising a characteristic acquisition and analysis module connected to said region merging module, and used to acquire and analyze at least one tumor characteristic from each said suspicious tumor tissue full region so as to recognize each said suspicious tumor tissue full region to be a tumor tissue region or non-tumor tissue region.

5. The ultrasonic imaging breast tumor detection and diagnostic system according to claim 4, wherein said characteristic acquisition and analysis module is used for classifying each said recognized tumor tissue region by using the BI-RADS (breast imaging reporting and data system) assessment category scale.

6. The ultrasonic imaging breast tumor detection and diagnostic system according to claim 5, further comprising a tumor marker module connected to said characteristic acquisition and analysis module, said tumor marker module providing a tumor map and adapted to mark all said recognized tumor tissue regions on said tumor map according to their distribution position in the breast.

7. The ultrasonic imaging breast tumor detection and diagnostic system according to claim 6, wherein said tumor marker module is used for marking all said recognized tumor tissue regions on said tumor map in different colors subject to the respective classified BI-RADS assessment categories.

8. The ultrasonic imaging breast tumor detection and diagnostic system according to claim 6, wherein said tumor marker module is used for calculating the position and size of each said recognized tumor tissue region based on the nipple position as a center.

9. The ultrasonic imaging breast tumor detection and diagnostic system according to claim 6, further comprising a user interface connected to said tumor marker module, said user interface comprising a tumor map display zone and being adapted to display said tumor map on said tumor map display zone.

10. The ultrasonic imaging breast tumor detection and diagnostic system according to claim 8, further comprising a user interface connected to said tumor marker module, said user interface comprising a tumor diagnosis zone adapted to display the diagnostic result of each said recognized tumor tissue region in a list, said diagnostic result comprising said BI-RADS assessment category of each said recognized tumor tissue region and the position and/or size of each said recognized tumor tissue region.

11. The ultrasonic imaging breast tumor detection and diagnostic system according to claim 10, wherein the diagnostic results of said recognized tumor tissue regions are listed in a predetermined order subject to the respective BI-RADS assessment categories, positions and/or sizes.

12. The ultrasonic imaging breast tumor detection and diagnostic system according to claim 1, further comprising a user interface connected to said image acquisition module, said user interface comprising a breast scanning site selection zone and an ultrasound imaging display zone, said breast scanning site selection zone comprising a plurality of scanning site selection components, said 3D breast ultrasound image of the corresponding breast site be displayed on said ultrasound imaging display zone via the click of said specific scanning site selection component.

13. An ultrasonic imaging breast tumor detection and diagnostic method, comprising the steps of:
acquiring a plurality of 3D breast ultrasound images;
cutting out a plurality of regions from said 3D breast ultrasound images using a 3D means shift algorithm;
acquiring the mean grayscale value (MGV) of each said region;
setting a plurality of groups for classifying the mean grayscale value (MGV) of each said region;
classifying each said region to the corresponding group subject to the mean grayscale value (MGV) of each said region; and
merging each of the regions of the darkest group with at least one adjacent region of the similar grayscale into a respective suspicious tumor tissue full region.

14. The ultrasonic imaging breast tumor detection and diagnostic method according to claim 13, further comprising the steps of:
acquiring at least one tumor characteristic from each said suspicious tumor tissue full region; and
analyzing said tumor characteristic of each said suspicious tumor tissue full region to recognize each said suspicious tumor tissue full region to be a tumor tissue region or non-tumor tissue region.

15. The ultrasonic imaging breast tumor detection and diagnostic method according to claim 14, further comprising the step of performing a benign and malignant classification on each said suspicious tumor tissue full region been recognized as a tumor tissue region by using the BI-RADS assessment-category scale.

16. The ultrasonic imaging breast tumor detection and diagnostic method according to claim 15, further comprising the step of marking all said recognized tumor tissue regions on a tumor map.

17. The ultrasonic imaging breast tumor detection and diagnostic method according to claim 16, wherein all said recognized tumor tissue regions are marked on a tumor map in different colors subject to the respectively classified BI-RADS assessment categories.

18. The ultrasonic imaging breast tumor detection and diagnostic method according to claim 16, further comprising the step of calculating the position and size of each said recognized tumor tissue region.
